(19) United States
(12) Patent Application Publication
(10) Pub. No.: US 2005/0169945 A1
(43) Pub. Date: Aug. 4, 2005

(54) Composition for protecting keratin material, process of making, uses thereof

(75) Inventor: Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/045,265

(22) Filed: Jan. 31, 2005

Related U.S. Application Data
(60) Provisional application No. 60/545,190, filed on Feb. 18, 2004.

(30) Foreign Application Priority Data
Jan. 29, 2004 (FR) 0400850

(51) Int. Cl. A61K 31/59; A61K 31/525; A61K 31/505; A61K 31/4415; A61K 31/51; A61K 31/455; A61K 31/355

(52) U.S. Cl. 424/400; 514/167; 514/251; 514/275; 514/300; 514/356; 514/458; 514/474; 514/690; 514/725; 514/276

(57) ABSTRACT

The present invention relates to a process for preparing a (cosmetic) composition useful for example for the treatment of keratin materials, in particular the skin and human keratin fibres such as the hair, to the compositions produced, and also to a process for treating keratin materials using this composition. The invention process percolates a fluid containing at least steam, at a pressure ranging from 3 to 30 bar, through at least one vitamin in solid or pasty form.

### REFERENCE TO PRIOR APPLICATIONS

This application claims priority to U.S. provisional application 60/545,190 filed Feb. 18, 2004, and to French patent application 0400850 filed Jan. 29, 2004, both incorporated herein by reference.

### FIELD OF THE INVENTION

The present invention relates to a process for preparing a (cosmetic) composition useful for example for the treatment of keratin materials, in particular the skin and human keratin fibres such as the hair, to the compositions produced, and also to a process for treating keratin materials using this composition.

More particularly, the present invention relates to a process for preparing a (cosmetic) composition for treating keratin materials, wherein it comprises a step of percolating fluid comprising at least steam, at a pressure ranging from 3 to 30 bar, through at least one vitamin in solid or pasty form.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In cosmetics, attempts are always being made to improve the cosmetic properties of keratin materials. Thus, it is known practice to use, in compositions for topical application, active agents, and in particular vitamins, for combating skin damage, and in particular the signs of ageing of the skin, for nourishing the skin and for giving the skin and the other keratin materials treated with these compositions all the benefits associated with these vitamins.

It is known that, in the course of the ageing process, various signs appear on human skin that are very characteristic of this ageing, in particular resulting in a change in the structure and function of the skin. The main clinical signs of skin ageing are in particular the following: the appearance of fine lines and then of deep wrinkles that increase with age, and disorganization of the “grain” of the skin, i.e. the microrelief is less uniform and has an anisotropic nature.

Moreover, the skin complexion is generally modified; it appears paler and yellower, which appears to be due essentially to disorganization of the microcirculation (less haemoglobin in the papillary dermis). Numerous coloured marks appear at the surface, which is due to impaired melanogenesis. Another clinical sign of ageing is the dry and coarse appearance of the skin, which is due essentially to more extensive desquamation; by diffracting light rays, these squamae also contribute to the slightly grey appearance of the complexion.

Thus, it is known practice to treat these signs of ageing by using cosmetic or dermatological compositions containing vitamins.

It is also known practice to treat keratin fibres with vitamins, when the fibres have been subjected to a reductive treatment, for instance permanent-waving, and/or to an oxidative treatment, for instance dyeing, or when they have been subjected to an external stimulus, for instance light or chlorine. By means of this treatment, the colour and sheen of the fibres are revitalized, and the smoothing of the fibres is improved.

It is, however, necessary to have compositions for the cosmetic treatment of keratin materials in which the solubilization of the vitamins is improved, and to increase the cosmetic effectiveness of the vitamins. In addition, vitamins are compounds that are unstable, in particular in the presence of light, of an oxidizing medium, such as air, and/or of water.

### DETAILED DESCRIPTION OF THE PREFERRED EMBODIMENTS

It has now been discovered, surprisingly, that by using a novel process for preparing a composition, it is possible to obtain, in a very short period of time, for example less than 2 minutes, compositions that are more or less concentrated in vitamins, according to need, in particular without preserving agent, making it possible to overcome the problems of solubility, of stability and of effectiveness disclosed above.

This process is simple to carry out. A fluid with a temperature preferably of greater than or equal to 30° C., preferably ranging from 30° C. to 150° C., and even more preferably ranging from 40° C. to 120° C., is passed, under pressure, for a very short period of time, for example of less than one minute, through at least one vitamin in solid or pasty form, preferably in solid form, and more preferably in pulverulent form.

It also enables the use, in anhydrous form, of vitamins which are unstable in aqueous compositions, either because they react with water or because they react in aqueous solution with compounds that do not react with them in an anhydrous composition.

The compositions prepared according to this process may have limited stability on storage, which is not a drawback in this case since the process leads to a ready-to-use composition intended to be used rapidly after its preparation, for example within 5 minutes of its preparation, in particular after cooling to a temperature that is cosmetically acceptable, preferably of less than 60° C. The composition may be used up to one week or more after its preparation, depending on the rate of degradation of the vitamin.

Given the very short preparation time, the cosmetic treatment compositions may be prepared “on demand” by mixing the active compounds according to the desired cosmetic properties.

According to another embodiment, the vitamins may be packaged in a ready-to-use device, and it is not necessary to determine beforehand the concentrations of the vitamins in solution, which limits the measuring errors by the user.

In addition, the process according to the invention makes it possible to avoid the use of multicompartment bottles, which makes the process particularly economical and safer for the user.

The composition thus obtained may be used alone or as a mixture with another composition.

An important advantage of this preparation process is the production of compositions that confer good cosmetic properties. In particular, the keratin materials treated with a composition obtained by means of the process according to the invention are protected, nourished and strengthened, and have a particularly pleasant visual appearance.

A subject of the invention is therefore a process for preparing a composition for the cosmetic treatment of keratin materials, comprising a step of percolating a fluid under a pressure of at least 3 bar through at least one vitamin in solid or pasty form.

Another subject of the invention is a composition that can be obtained by means of the process according to the invention.

A subject of the invention is also the use, in particular cosmetic use, of the composition obtained according to the process of the invention, as an agent for the cosmetic treatment of keratin materials.

Finally, a subject of the invention is a packaging device for implementing the preparation process of the present invention.

Other subjects, characteristics, aspects and advantages of the invention will emerge even more clearly on reading the further nonlimiting description and the examples that follow.

According to the invention, the process for preparing a composition for the cosmetic treatment of keratin materials comprises a step of percolating a fluid, preferably at a temperature at least equal to 30° C., better still ranging from 30° C. to 150° C., better still from 40° C. to 120° C., under a pressure of at least 3 bar (3×105 Pa), through at least one vitamin in solid or pasty form.

Percolation is a movement of fluid through a saturated porous medium, allowing passage of the fluid due to the action or effect of pressure.

The fluid may comprise steam optionally accompanied by at least one of liquid water and one or more cosmetically acceptable liquid and/or gaseous solvents, in particular organic solvents. Preferably, the fluid comprises at least steam accompanied by liquid water.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether, diethylene glycol monomethyl ether and monoethyl ether, and also aromatic alcohols, for instance benzyl alcohol or phenoxyethanol, and mixtures thereof.

The expression “water-insoluble compound” is intended to mean any compound which, at a concentration greater than or equal to 0.1% by weight in water at 25° C., does not form, to the naked eye, a transparent isotropic solution.

The vitamin is in solid form or in pasty form, preferably in solid form, and even more preferably in pulverulent form.

For the purpose of the present invention, the term “pasty form” is intended to mean a consistency that is intermediate between a solid phase and a liquid phase. The viscosity of this pasty phase is preferably greater than 0.1 Pa.s, and even more preferably greater than 1 Pa.s, at 25° C. with a shear rate of 10 s-1.

For the purpose of the present invention, the term “vitamin” is intended to mean vitamins, their derivatives such as esters, including glycerides, glucosides, salts and ethers; and their precursors.

The term “keratin materials” is intended to mean the skin, the scalp, the lips and/or the integuments such as the nails, the eyelashes, the eyebrows and the hair.

The process of the present invention may be carried out using a standard device for generating a pressurized fluid, at a temperature preferably of greater than or equal to 30° C., and even more preferably ranging from 30° C. to 150° C. Such a device comprises a pressure-resistant chamber equipped with a thermal block, and also a circuit for conveying the fluid produced to the vitamin.

According to another embodiment, the device comprises a reservoir of liquid(s) and also a pump for conveying the liquid(s) to the chamber.

The liquid contained in the reservoir is either water, or a cosmetically acceptable solvent or a mixture of several cosmetically acceptable solvents, or alternatively a mixture of water and of one or more cosmetically acceptable solvents. The liquid preferably comprises at least water, and even more preferably it is water.

A device that is particularly useful for carrying out the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to a particular embodiment of the invention, the percolation step is carried out with a fluid at a temperature of greater than 30° C., preferably of between 30° C. and 150° C., under a pressure of between 3 and 30 bar, or of at least 4 bar, preferably greater than 10 bar, and most particularly between 10 and 30 bar.

The vitamin(s), in solid or pasty form, may be used directly in the device generating the pressurized fluid, in a container intended for this purpose. It (they) may also be packaged in a particular packaging device, of the single-dose type, comprising a closed housing delimited by at least one wall that is at least partially permeable to a fluid under a pressure of at least 3 bar. Such devices are, for example, described in patent applications WO 00/56629, EP 512470, U.S. Pat. No. 5,897,899 or WO 99/03753. These packaging devices are generally airtight, watertight and/or lightproof.

According to a particular embodiment, the housing is delimited by two sealed sheets. According to another embodiment, the housing is delimited by a tray closed with a lid.

These devices may be manufactured from woven or nonwoven plastic or plant materials, for example from cellulose, from metal such as aluminium or from composite materials. Such devices are described, for example, in patent applications WO 00/56629, EP 512470, U.S. Pat. No. 5,897,899 or WO 99/03753.

The vitamins used according to the invention are preferably chosen from vitamin A, vitamin B3, vitamin B5, vitamin B9, vitamin C, vitamin E, vitamin F, vitamin H, their derivatives, their precursors and their mixtures.

Vitamin C

Ascorbic acid, or vitamin C stimulates the synthesis of connective tissue, and in particular of collagen, strengthens the defences of skin tissue against outside attacks such as ultraviolet radiation and pollution, compensates for vitamin E deficiency of the skin, depigments the skin, and has a free-radical scavenger function. The latter two properties make it an excellent candidate as a cosmetic or dermatological active agent for combating and/or preventing ageing of the skin. Vitamin C also makes it possible to protect the hair against oxidative phenomena, in particular of free-radical origin.

Vitamin C corresponds to ascorbic acid which is generally in the L form, since it is usually extracted from natural products. The ascorbic acid derivatives include, more particularly, its salts, such as in particular sodium ascorbate, magnesium ascorbyl phosphate or sodium ascorbyl phosphate; its esters, for instance especially its esters such as ascorbyl acetate, tocopherol nicotinate, ascorbyl palmitate and ascorbyl propionate, or its sugars, such as in particular glycosylated ascorbic acid, tocopherol succinate, magnesium ascorbyl phosphate, salts such as sodium ascorbate, and mixtures thereof.

Vitamins B3 and B5 can react in order to treat or prevent skin ageing, and also to remove the oily appearance of the skin, to make the texture of the skin and/or the size of the pores of the skin uniform, and/or to treat oily skin. They also make it possible to obtain shiny hair that has a healthy appearance, and to stimulate hair growth or to slow down hair loss.

Vitamin B3

Vitamin B3, also called vitamin PP, is a compound of formula 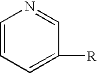
in which R may be —CONH2 (niacinamide), —COOH (nicotinic acid or niacin), or CH2OH (nicotinyl alcohol), —CO—NH—CH2-COOH (nicotinuric acid) or —CO—NH—OH (niconityl hydroxamic acid).

Vitamin B3 derivatives that may be mentioned include, for example, nicotinic acid esters such as tocopherol nicotinate, amides derived from niacinamide by substitution of the hydrogen groups of —CONH2, products from reaction with carboxylic acids and amino acids, esters of nicotinyl alcohol and of carboxylic acids such as acetic acid, salicyclic acid, glycolid acid or palmitic acid. Mention may also be made of the following derivatives: 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine, methyl nicotinate and sodium nicotinate.

Other vitamin B3 derivatives that may also be mentioned include its inorganic salts, such as chlorides, bromides, iodides or carbonates, and its organic salts, such as the salts obtained by reaction with carboxylic acids, such as acetate, salicylate, glycolate, lactate, malate, citrate, mandelate, tartrate, etc.

Vitamin B5

As vitamin B5, use may be made of panthenol or penthenyl alcohol or 2,4-dihydroxy-N-(3-hydroxypropyl)-3,3-dimethylbutanamide, in its various forms: D-panthenol, DL-panthenol, and its derivatives and analogues, such as calcium pantothenate, panthetine, pantotheine, dipenthenyl ethyl ether, pangamic acid, pyridoxine or pantoyl lactose, and natural compounds containing vitamin B5, such as royal jelly.

Use may also be made of vitamin B9, or folic acid.

Vitamin D

Vitamin D is an essential vitamin for the prevention and treatment of mineralization deficiencies in cartilage (rickets) and in bone (osteomalacia), and even of certain forms of osteoporosis in elderly individuals. However, these functions extend well beyond the regulation of bone metabolism and of calcium homeostasis. Among these, mention may be made of its actions on cell proliferation and on cell differentiation and immune defence control. When applied topically, vitamin D and its analogues make it possible in particular to treat skin disorders such as, for example, psoriasis and signs of ageing.

As vitamin D, mention may be made of 1α,25-dihydroxyvitamin D3 and its analogues, and also the vitamin D analogues such as those described in document WO-A-00/26167, for instance:
3-hydroxymethyl-5-{2-[3-(5-hydroxy-5- or 6-methylhexyl)-phenyl]vinyl}phenol,
3-[3-(5-hydroxy-1,5-dimethylhexyl)phenoxymethyl]-5-hydroxymethylphenol,
6-[3-(3,4-bishydroxymethylbenzyloxy)phenyl]-2-methylhepta-3,5-dien-2-ol,
6-[3-(3,4-bishydroxymethylbenzyloxy)phenyl]-2-methyl-hexan-2-ol,
6-[3-(3,4-bishydroxymethylphenoxymethyl)phenyl]-2-methylheptan-2-ol,
7-[3-(3,4-bishydroxymethylphenoxymethyl)phenyl]-3-ethyloctan-3-ol,
5-{2-[4-(5-hydroxy-5-methylhexyl)phenyl]vinyl or -ethyl}benzene-1,3-diol,
5-{2-[3- or 4-(6-hydroxy-6-methylheptyl)phenyl]vinyl}benzene-1,3-diol,
5-{2-[3- or 4-(6-hydroxy-6-methylheptyl)phenyl]ethyl}-benzene-1,3-diol,
2-hydroxymethyl-4-{2-[3- or 4-(5-hydroxy-5-methylhexyl)-phenyl]vinyl}phenol,
2-hydroxymethyl-4-{2-[3- or 4-(6-hydroxy-6-methylheptyl)-phenyl]vinyl}phenol,
2-hydroxymethyl-4-{2-[3- or 4-(5-hydroxy-5-methylheptyl)-phenyl]ethyl}phenol,
2-hydroxymethyl-4-{2-[3- or 4-(6-hydroxy-6-methylheptyl)-phenyl]ethyl}phenol,
2-hydroxymethyl-5-{2-[4-(5-hydroxy-5-methylhexyl)phenyl]vinyl}phenol,
6-[3-(3,4-bishydroxymethylbenzyloxy)phenyl]-2-methyl-heptan-2-ol,
4-[3-(5-hydroxy-1,5-dimethylhexyl)phenoxymethyl]-2-hydroxymethylphenol,
6-{3- or 4-[2-(3,4-bishydroxymethylphenyl)vinyl]phenyl}-2-methylhexan-2-ol,
7-{4-[2-(3,4-bishydroxymethylphenyl)vinyl]phenyl}-2-methylheptan-2-ol,
5-{2-[3-(6-hydroxy-6-methylheptyl)phenyl]-1-methylvinyl) benzene-1,3-diol,
5-{2-[3-(5-hydroxy-5-methylhexyl)phenyl]vinyl}benzene-1,3-diol,
5-[3-(6-hydroxy-6-methylheptyl)phenoxymethyl]benzene-1,3-diol,
5-{2-[3-(7-hydroxy-7-methyloct-1-enyl)phenyl]vinyl}-benzene-1,3-diol,
5-{2-[3-(7-hydroxy-7-methyloctyl)phenyl]vinyl}benzene-1,3-diol,
4-{2-[3-(6-hydroxy-6-methylheptyl)phenyl]vinyl}benzene-1,2-diol,
3-{2-[3-(6-hydroxy-6-methylheptyl)phenyl]vinyl) phenol,
6-{3-[2-(3,5-bishydroxymethylphenyl)vinyl]phenyl}-2-methylhexan-2-ol,
3-{2-[3-(7-hydroxy-7-methyloctyl)phenyl]vinyl}phenol,
7-{3-[2-(3,5-bishydroxymethylphenyl)vinyl]phenyl}-2-methylheptan-2-ol,
7-{3-[2-(3,4-bishydroxymethylphenyl)vinyl]phenyl}-2-methylheptan-2-ol,
7-{3-[2-(4-hydroxymethylphenyl)vinyl]phenyl}-2-methylheptan-2-ol,
4-{2-[3-(7-hydroxy-7-methyloct-1-enyl)phenyl]vinyl}benzene-1,2-diol,
7-[3-(3,4-bishydroxymethylphenylethynyl)phenyl]-2-methylheptan-2-ol,
5-{2-[3-(6-hydroxy-6-methylhept-1-enyl)phenyl]vinyl}benzene-1,3-diol,
5-{2-[3-(7-ethyl-7-hydroxynon-1-enyl)phenyl]vinyl}benzene-1,3-diol,
5-{2-[3-(7-hydroxy-1-methoxy-1,7-dimethyloctyl)phenyl]vinyl}benzene-1,3-diol,
5-{2-[3-(6-hydroxy-1-methoxy-1,6-dimethylheptyl)phenyl]vinyl}benzene-1,3-diol,
5-{2-[3-(5-hydroxypentyl)phenyl]vinyl}benzene-1,3-diol,
5-{2-[3-(5-hydroxy-6-methylheptyl)phenyl]vinyl}benzene-1,3-diol,
5-{2-[3-(6-hydroxy-7-methyloctyl)phenyl]vinyl}benzene-1,3-diol,
5-{2-[3-(5-hydroxy-6-methylhept-1-enyl)phenyl]vinyl}benzene-1,3-diol,
5-{2-[3-(6-hydroxy-7-methyloct-1-enyl)phenyl]vinyl}benzene-1,3-diol,
5-{2-[3-(1,6-dihydroxy-1,6-dimethylheptyl)phenyl]vinyl}benzene-1,3-diol,
5-{2-[3-(6-hydroxy-1,6-dimethylhept-1-enyl)phenyl]vinyl}benzene-1,3-diol.

Vitamin F

Vitamin F makes it possible to combat in particular dryness of the skin. It also makes it possible to strengthen the hair and to stimulate growth thereof or to slow down loss thereof.

Vitamin F is a mixture of essential fatty acids, i.e. unsaturated acids having at least one double bond, such as linoleic acid or 9,12-octadecadienoc acid and its stereoisomers, linolenic acid in the α-form (9,12,1 5-octadecatrienoic acid) or in the γ-form (6,9,12-octadecatrienoic acid) and their stereoisomers, arachidonic acid or 5,8,11,14-eicosatetraenoic acid and its stereoisomers.

Vitamin F or mixtures of unsaturated acids having at least one double bond, and in particular mixtures of linoleic acid, of linolenic acid and of arachidonic acid, or compounds containing it, and in particular oils of plant origin containing it, such as for example jojoba oil, can be used in the composition of the present invention. It is also possible to use vitamin F in the form of derivatives, and in particular in the form of esters such as esters of sugars and of vitamin F.

Vitamin H, also called biotin, can also be used. It can be used to harden the nails.

The vitamin(s) is (are) preferably chosen from vitamin A, vitamin C, vitamin B5, vitamin E and vitamin F.

The vitamin(s) may be used as a mixture with one or more solid or pasty, and preferably pulverulent, adjuvants. Once the percolation has finished, these adjuvants may remain in the percolator. The adjuvants may be chosen from clays, salts, anionic, cationic, nonionic, amphoteric or zwitterionic surfactants, natural or synthetic thickeners, glass beads, silica, nylon, waxes, pigments, alumina, titanium dioxide, zeolites, poly(methyl methacrylate) (PMMA), chitosan, maltodextrin, cyclodextrin, mono- or disaccharides such as glucose, sucrose, sorbitol or fructose, zinc oxide, zirconium oxide, resin particles, for instance silicone or silica beads, talc, polyaspartic acid, borosilicates, in particular calcium borosilicate, polyethylene, cotton, polytetrafluoroethylene (PTFE), cellulose and its derivatives, superabsorbent compounds, magnesium carbonate or calcium carbonate, optionally modified starch, corn seeds, polydimethylsiloxane gums, polyacrylamide, porous hydroxyapatite, silk, collagen, sawdust, wrack powder, meals or extracts of wheat, of rice, of pea, of lupin, of soybean or of barley, crosslinked polyvinylpyrrolidone, calcium alginate, active charcoal, and poly(vinylidene chloride/acrylonitrile) particles, in particular those sold under the general name “Expancel®” by the company Akzo Nobel under the particular Expancel references “Expancel® WE” or “Expancel® DE”, and mixtures thereof.

When one or more adjuvants is (are) present, the vitamin(s) is (are) preferably present in an amount ranging from 0.5 to 99% by weight, better still from 1 to 80% by weight, and even more preferably from 2 to 60% by weight relative to the total weight of vitamin(s) and adjuvants.

The plants or plant extracts used, and through which the pressurized fluid passes, may be subjected, before percolation, to a treatment such as roasting, cryogrinding, or lyophilization.

The composition for the cosmetic treatment of keratin materials obtained according to the process of the invention contains, besides the vitamin(s) and the component(s) of the fluid, i.e. water and/or the cosmetically acceptable solvent(s), optionally all or some of the adjuvant(s) present in the solid or pasty mixture.

The invention also relates to a composition that can be obtained by means of the process according to the invention, it being possible for the composition to be free of preserving agents.

Using the preparation process of the invention, a composition for the cosmetic treatment of keratin materials is obtained, which may be applied directly to the keratin materials, or which may be mixed with a cosmetically acceptable medium, or alternatively at least one additive conventionally used in cosmetics may be added thereto by an operator. At least two compositions obtained by means of the process of the invention may also be mixed together. The composition for the cosmetic treatment of keratin fibres optionally resulting from the mixture(s) and/or addition(s) indicated above will be referred to hereinafter as the final cosmetic treatment composition or final composition.

A particular embodiment of the invention comprises applying the composition obtained by means of a device that does not require any human intervention and that is optionally equipped with a cooling means.

Another particular embodiment of the invention comprises ingesting the cosmetic treatment composition obtained according to the process of the invention, when no toxicity problem is known in the art.

The amount of vitamins present in the final cosmetic treatment composition obtained by means of the process of the present invention is not limited and may generally be between 0.001 and 50% by weight, approximately, of the total weight of the final cosmetic treatment composition, preferably between 0.005 and 30%, and even more preferably between 0.01 and 20%.

When the cosmetic composition obtained by means of the process of the present invention is mixed with a cosmetically acceptable medium, the medium generally consists of water or of a mixture of water and of at least one organic solvent to solubilize the compounds which would not be sufficiently soluble in water. Examples of organic solvents that may be mentioned include, for example, C1-C4 lower alcohols, such as ethanol and isopropanol; polyols and polyol ethers, for instance 2-butoxyethanol, propylene glycol, propylene glycol monomethyl ether, and diethylene glycol monoethyl ether and monomethyl ether, and also aromatic alcohols, for example benzyl alcohol or phenoxyethanol, and mixtures thereof.

The solvents are preferably present in proportions preferably of between 1 and 40% by weight relative to the total weight of the final composition, and even more preferably of between 5 and 30% by weight.

At least one additive conventionally used in cosmetics may also be added to the cosmetic treatment compositions obtained according to the process of the present invention. By way of examples of such additives, mention may be made of anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrating agents, sequestering agents, fragrances, buffers, dispersants, conditioning agents such as, for example, silicone oils, film-forming agents, ceramides, preserving agents, opacifiers, but also oils, waxes, gums, and coloured or pearlescent pigments.

The above additives are generally present in an amount for each of them of between 0.01 and 20% by weight relative to the weight of the final composition.

Of course, those skilled in the art will take care to select this or these optional compounds such that the advantageous properties intrinsically associated with the cosmetic composition in accordance with the invention are not, or are not substantially, adversely affected by the addition(s) envisaged.

The pH of the final composition is generally between 3 and 12, and preferably between 5 and 11. It can be adjusted to the desired value by means of acidifying or basifying agents normally used in cosmetics, or alternatively using standard buffer systems.

Among the acidifying agents that may be mentioned, for example, are mineral or organic acids such as hydrochloric acid, orthophosphoric acid, sulphuric acid, carboxylic acids such as acetic acid, tartaric acid, citric acid and lactic acid, and sulphonic acids.

Among the basifying agents that may be mentioned, for example, are aqueous ammonia, alkaline carbonates, alkanolamines such as mono-, di- and triethanolamines and derivatives thereof; sodium hydroxide, potassium hydroxide and compounds of formula (II) below: 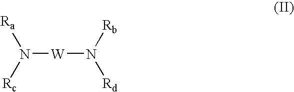
in which W is a propylene residue that is optionally substituted with a hydroxyl group or a C1-C4 alkyl radical; Ra, Rb, Rc and Rd, which may be identical or different, represent a hydrogen atom, a C1-C4 alkyl radical or a C1-C4 hydroxyalkyl radical.

The final cosmetic treatment composition may be in various forms, such as in the form of liquids, creams, gels or mascara, or in any other form that is suitable for treating keratin materials, and in particular keratin fibres, and the skin. In particular, the final cosmetic treatment composition may be a lipstick containing vitamins A and E or derivatives thereof, having a protective effect and an effect against drying out, and a lipstick containing vitamin C for a healing effect against chapping and cracking. The final composition may also be a foundation for colouring the skin and/or protecting it.

The present invention also relates to a process for the cosmetic treatment of keratin materials, comprising the preparation of a cosmetic treatment composition according to the process as defined above, and its application to the keratin materials, for example by means of an operator or by means of a device that does not require any human intervention. The application time may preferably range between 15 seconds and 1 hour.

Before application, the cosmetic treatment composition obtained according to the process of the invention may be mixed with a cosmetically acceptable medium and/or with one or more additives conventionally used in cosmetics, as described above.

Another embodiment comprises preparing at least two cosmetic treatment compositions according to the process of the invention, in mixing them together, and in optionally adding a cosmetically acceptable medium and/or one or more additives conventionally used in cosmetics, as described above, and then in applying the final composition obtained to the keratin materials.

The examples below are intended to illustrate the present invention, but not limit it.

### EXAMPLE 1

### Preparation of a Conditioner

The following ingredients are mixed together:
2 g of powdered vitamin C sold by the company Roche Vitamins
2 g of ammonium bicarbonate
1 g of maltodextrin

This mixture of 5 g of powder is placed in an espresso machine. Steam is then passed through until a composition (A) having a final volume of 50 ml is obtained.

One part by weight of an aqueous composition (B) containing 1% by weight of hydroxyethylcellulose can then be added to two parts by weight of composition (A), in order to facilitate application.

After applying it to the hair, leaving it on for two minutes, and rinsing, smooth and strengthened hair is obtained.

### EXAMPLE 2

### Preparation of an Anti-Wrinkle Gel for the Skin

5 g of powdered vitamin C sold by the company Roche Vitamins are prepared.

This powder, which is in a container intended to receive a solid compound, is placed in an espresso machine. Steam produced by the machine, under a pressure of 3 bar, then passes through the powder. The composition obtained is introduced, just before application to the skin, into a gel buffered at pH 4, containing:

Phase A

| | |
|---|---|
| Carrageenan | 1.5 g |
| Lubrajel (gelling agent) | 28.5 g |
| Preserving agent | 0.2 g |
| Deionized water qs | 95 g |
| Sodium hydroxide | 0.5 g |

Phase B

| | |
|---|---|
| Deionized water | 4.8 g |
| Germall 115 | 0.3 g |
| Dequest 2046 | 0.1 g |
| Aloe extract | 0.5 g |

The gel is produced conventionally by separately preparing, by simple mixing, phases A and B respectively at 70° C. and 40° C., then adding phase B to phase A with stirring at 40° C. and allowing the entire mixture to cool, with slow stirring, to ambient temperature. The composition obtained allows smoothing of the face and good moisturizing of the skin.

The above written description of the invention provides a manner and process of making and using it such that any person skilled in this art is enabled to make and use the same, this enablement being provided in particular for the subject matter of the appended claims, which make up a part of the original description and including a process for preparing a composition, comprising percolating fluid comprising at least steam, at a pressure ranging from 3 to 30 bar, through at least one vitamin, the compositon prepared, and a process using the composition for treating keratin fibers.

As used herein, the phrases “selected from the group consisting of,” “chosen from,” “selected from,” and the like include mixtures of the specified materials.

Where compounds are described as, e.g, “Vitamin_ and its derivatives” etc., an alternate is “Vitamin_ and Vitamin_ compounds” where Vitamin_ derivatives and compounds share a common core/structure with Vitamin_. One of ordinary skill in the art knows how to identify derivatives and compounds of compound (e.g., Vitamin) X based on the structure of X and the similarity in structure of the derivatives and compounds.

Where a term is enclosed by parentheses it is an optional modifier. For example, the term “(cosmetic) composition” describes both a composition in general and a cosmetic composition.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating a fluid comprising steam, under a pressure of at least 3 bar, through at least one vitamin in solid or pasty form.

2. The process according to claim 1, wherein the vitamin(s) is (are) chosen from vitamin A, vitamin B3, vitamin B5, vitamin B9, vitamin C, vitamin E, vitamin F, vitamin H, their derivatives, their precursors, and mixtures thereof.

3. The process according to claim 2, wherein the vitamin(s) is (are) chosen from vitamin A, vitamin C, vitamin B5, vitamin E, vitamin F, and mixtures thereof.

4. The process according to claim 1, wherein the vitamin in solid or pasty form is present as a mixture with at least one adjuvant.

5. The process according to claim 4, wherein the adjuvant is chosen from clays, salts, anionic, nonionic, cationic or zwitterionic surfactants, natural or synthetic thickeners, optionally modified starch, glass beads, silica, nylon, alumina, titanium dioxide, zeolites, poly(methyl methacrylate) (PMMA), chitosan, maltodextrin, cyclodextrin, mono- or disaccharides, zinc oxide, zirconium oxide, resin particles, talc, polyaspartic acid, borosilicates, in particular calcium borosilicate, polyethylene, cotton, polytetrafluoroethylene (PTFE), cellulose and its derivatives, superabsorbent compounds, magnesium carbonate or calcium carbonate, corn seeds, polydimethylsiloxane gums, polyacrylamide, porous hydroxyapatite, silk, collagen, sawdust, wrack powder, meals or extracts of wheat, of rice, of pea, of lupin, of soybean or of barley, crosslinked polyvinylpyrrolidone, calcium alginate, active charcoal, poly(vinylidene chloride/acrylonitrile) particles and mixtures thereof.

6. The process according to claim 4, wherein the vitamin(s) is (are) present in an amount ranging from 0.5 to 99% by weight relative to the total weight of vitamin(s) and adjuvant(s).

7. The process according to claim 1, wherein the percolation step is carried out with a fluid under a pressure of between 3 and 30 bar.

8. The process according to claim 7, wherein the percolation step is carried out with a fluid under a pressure of at least 10 bar.

9. The process according to claim 1, wherein the fluid further comprises liquid water.

10. The process according to claim 1, wherein the fluid further comprises one or more cosmetically acceptable liquid and/or gaseous organic solvents.

11. A composition obtained by percolating fluid comprising at least steam, at a pressure ranging from 3 to 30 bar, through at least one vitamin.

12. The composition according to claim 11, wherein it is free of preserving agent.

13. A process for treating keratin material, wherein the composition of claim 11 is applied to keratin material.

14. The process according to claim 13, wherein the composition is applied to the keratin material by a device that does not require any human intervention.

15. The process according to claim 13, wherein, before application, the composition is mixed with a cosmetically acceptable medium and/or with one or more additives used in cosmetics.

16. The process according to claim 13, wherein, before application, the composition is mixed with another, different such composition and the mixture is applied to the keratin material.

17. A device for packaging a composition, comprising a closed housing delimited by at least one wall that is at least partially permeable to fluid at a pressure of at least 3 bar, the composition containing at least one vitamin in solid or pasty form.

18. The device according to claim 17, wherein the housing is delimited by two sealed sheets.

19. The device according to claim 17, wherein the housing is delimited by a tray closed with a lid.
